(19) United States
(12) Patent Application Publication
(10) Pub. No.: US 2010/0291503 A1
(43) Pub. Date: Nov. 18, 2010

(54) Tongue block

(76) Inventors: J. Richard Shih, San Diego, CA (US); Stephanie Tze-Fan Shih, San Diego, CA (US)

Correspondence Address: J. Richard Shih (Jeng-Kang Richard Shih), 14616 La Plata, San Diego, CA, 92127, US

(21) Appl. No.: 12/653,203

(22) Filed: Dec. 10, 2009

Related U.S. Application Data
(63) Continuation-in-part of application No. 10/697,932, filed on Oct. 31, 2003, now abandoned.

(51) Int. Cl. A61C 19/00; A61B 1/247

(52) U.S. Cl. 433/31; 433/140; 433/29

(57) ABSTRACT

The invention is a device for use as a prop to hold the mouth open which consists essentially of a bite block with an elongated portion that is deflected and to provide a physiologically shaped elongated portion extends along the buccal border of the tongue to form a tongue depressor and light reflector as the bite block is held in position. The retaining arm, tongue guiding hole and the flange retain the patient's tongue in the bottom cavity of the patient's mouth.

This application is a continuation-in-part of U.S. Ser. No. 10/697,932, filed Oct. 31, 2003, which is now abandoned.

### FIELD OF THE INVENTION

The present invention relates to an dental/medical device.

### BACKGROUND OF THE INVENTION

The subject invention is a prop for holding the mouth open to be used during oral surgery and during dental treatment and repair. The device is particularly valuable when caring for an uncooperative or incompetent patient under sedation, which will reduce the trauma or cutting tongue during the treatment procedure. The invention is also helpful as a tool to suppress tongue position when taking CAD/CAM digital impression, which greatly reduces the chair time for such a procedure.

### DESCRIPTION OF THE PRIOR ART

Many prop devices, with or without suction means, are available to the dentist and oral surgeon. However, an adaptation of the relatively simple prop disclosed in U.S. Pat. No. 2,220,674 continues to be used. That patent discloses and claims a device with a cheek-engaging convex portion having a pair of spaced diverging trough portions that formed a U-shaped trough section on which the teeth rested. Devices fitting this general description continue to be sold and used. They are effective for general dental procedure but can not effectively to prevent tongue cutting when patients are sedated.

U.S. Pat. No. 3,924,333 discloses a dental appliance for evacuating debris and liquid from the mouth. The device is a bite block with an opening in the transverse direction through the block to permit passage of a tube to provide for evacuation of the oral cavity. The device is used with an attached dam. The design is not for tongue blocking.

U.S. Pat. No. 4,167,814 discloses a bite block having a means for attachment of a suction tube that bends to provide a U-shaped portion with suction holes to evacuate fluid from the oral cavity. This appliance is not effective to block the tongue out of surgeon's instrumentation.

U.S. Pat. No. 4,192,071 discloses a dam connected to a bite block through which there passes a tube for suctioning the oral cavity. The device is quite complex and do not have concave mirror surface to reflect the light.

U.S. Pat. No. 4,975,057 discloses a bite block with openings to admit suction tubing. The device has an aperture in the transverse direction from the exterior of the bite block, through the block into the oral cavity. This appliance is not designed for blocking the tongue.

U.S. Pat. No. 5,466,153 discloses a bite block with handle design. This alliance is not designed to block the tongue.

U.S. Pat. No. 6,655,960 B2 disclosed a tongue suppressing bite block adaptable to varying mouth and tongue sizes. Not physiologically shaped and has no concave surface to reflect light into oral cavity.

Because of CAD/CAM technology is getting popular and intra-oral digital impression procedure has been performed as a routine in-office procedure, an effective tongue blocking device is now in need to save chair time of dentist. The current invention is believed to provide a great tool to make intra-oral digital impression easier.

### BRIEF DESCRIPTION OF THE FIGURES

FIG. 1 is a front medial view of the prop device when the device is in place. This view shows the concave reflective surface (21) and the tongue guiding aperture (22) for “tongue escape”.

FIG. 2 is a view of the block from the aspect which is front lateral when the device is in place with CAD/CAM impression camera (23). This view shows how the tongue tip (27) being guided into the “tongue escape” aperture.

FIG. 3 is a top view of the prop when the block is in place between the jaws to show the reflected concave surface (21) and the convex surface (24) on the back.

FIG. 4 is a posterior view of the block that shows the relationship between tongue guiding convex surface (24) and the tongue guiding escape aperture (22).

FIG. 5 shows a front view of the bite block with a mirroring surface (25), which is made onto the concave surface (21).

FIG. 6 is a medial view of the prop device showing the de-attachable LED light (26) in use with the device

FIG. 7 shows a kit for use in administering dental care.

### DETAILED DESCRIPTION OF THE INVENTION

The purpose of the invention is to provide an improved device for supporting the jaws in the open position during dental surgery and treatment in a manner that will facilitate use of instruments by the dental practitioner and will protect the patient's tongue by instruments in the patient's mouth during dental procedures while patient is sedated and tongue get enlarged because of sedation. The device is simple and economical enough to occasion wide use. Because the device is inexpensive, it can be sold as a disposable unit. Use of such disposable devices avoids expense of cleaning and spread of infection.

The device of the invention consists essentially of a bite block with an elongated portion that extends from the anterior aspect of the block so that the elongated portion is deflected to provide a physiological shape elongation and flange to act as tongue blocking device. The u-shape physiologically curved elongated portion extends around the inside the dentition from bite block to the posterior region of the space between tongue and teeth. Because of the shape of concave surface (21) can gather nature light into the oral cavity, the view of the dental practitioner is better when mirror surface feature is added to the tongue block or when an illuminated device such as LED light source (26) is added to the device. The device is feature a physiological “tongue escape” hole (22) for tongue to rest and secure into the hole when bite block is in place. The force of convex side (24) of the tongue block will push the tongue to rest into the “tongue escape” and secure the tongue in place from unwanted movement.

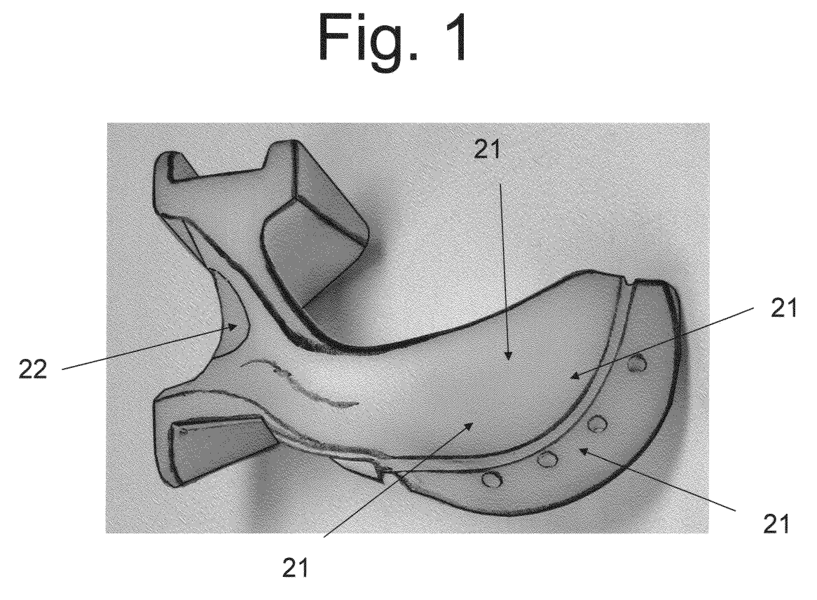

Referring to the drawings, FIG. 1 is a view of the device from the medial aspect, that is, the view from the side that is positioned against the tongue when the block is in place.

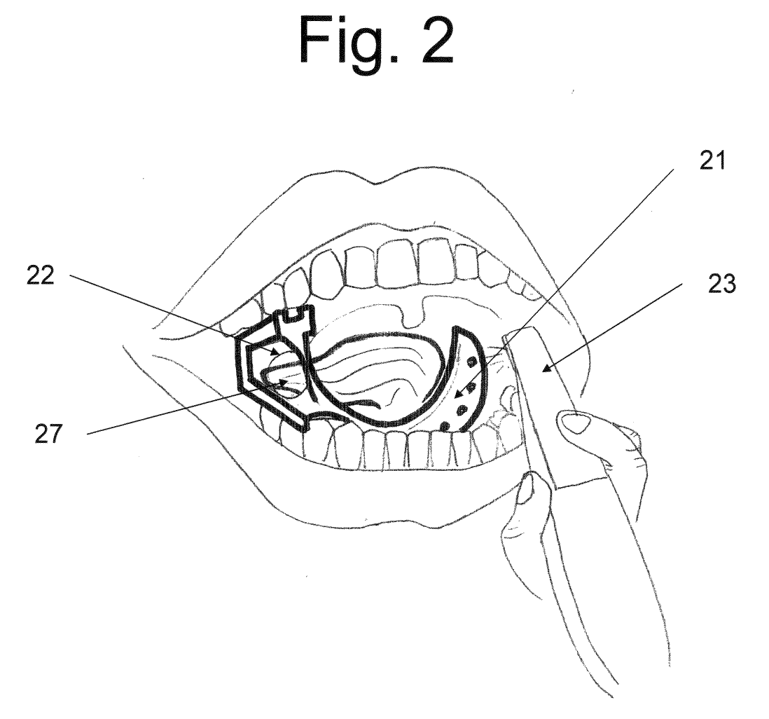

FIG. 2 is a view of the block from the front lateral aspect, that is, the view from the side that is positioned against the cheek when the block is in use with digital impression, which shows the tongue block is helping the impression taking procedure to actively push away the tongue out of the path of digital impression.

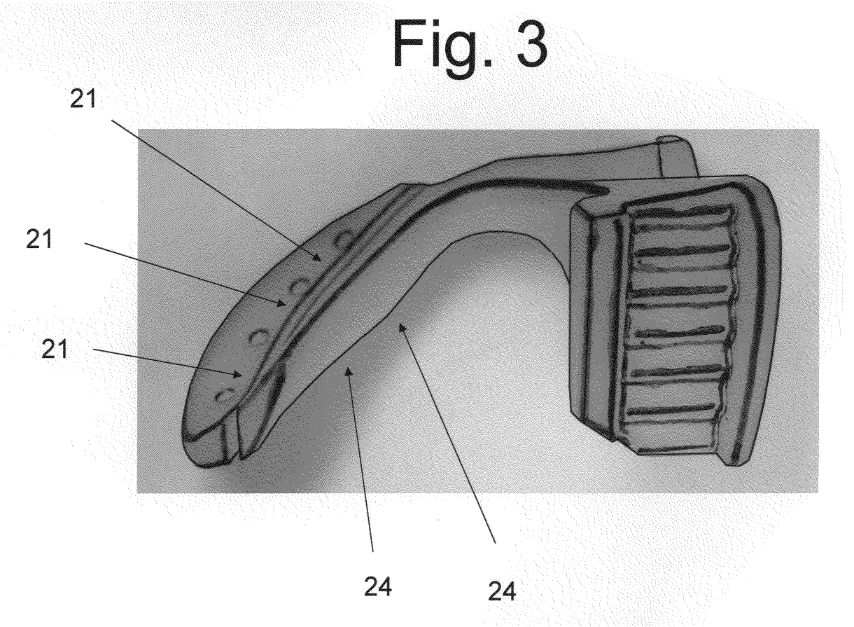

FIG. 3 is a top view of the block that shows the extension flange design. The ridges hold the teeth against the bite surfaces. The concaved reflective area is facing outside (teeth side) and the convex surface is facing inside (tongue side)

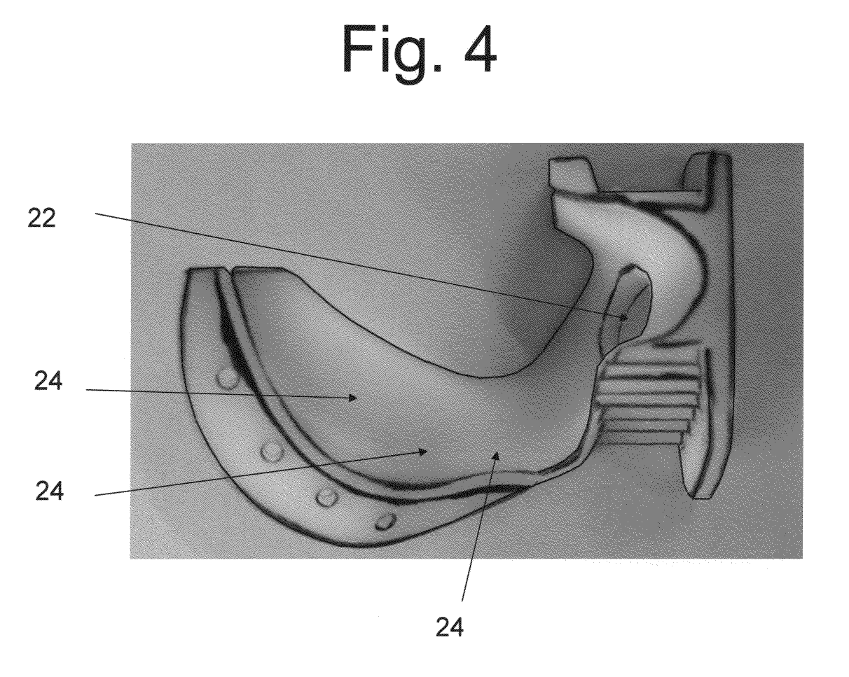

FIG. 4 provides a posterior view of the block that shows the relationship between convex surface (24) and the tongue guiding groove and the escape aperture (22) behind the device.

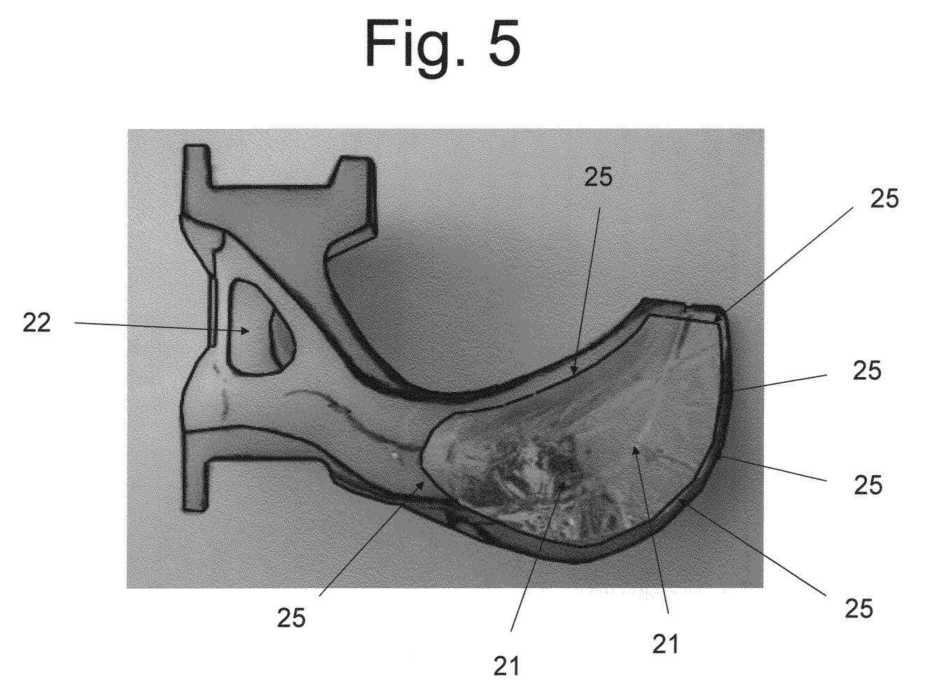

FIG. 5 depicts the block from front view with mirror surfaces (25) on the concave surface (21) to increase light gathering ability.

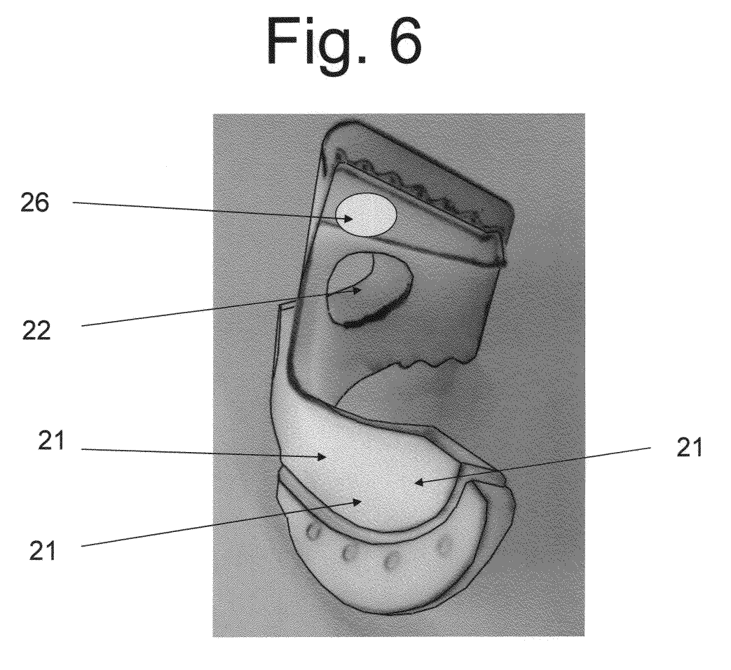

FIG. 6 depicts the device from medial view showing the LED light in place.

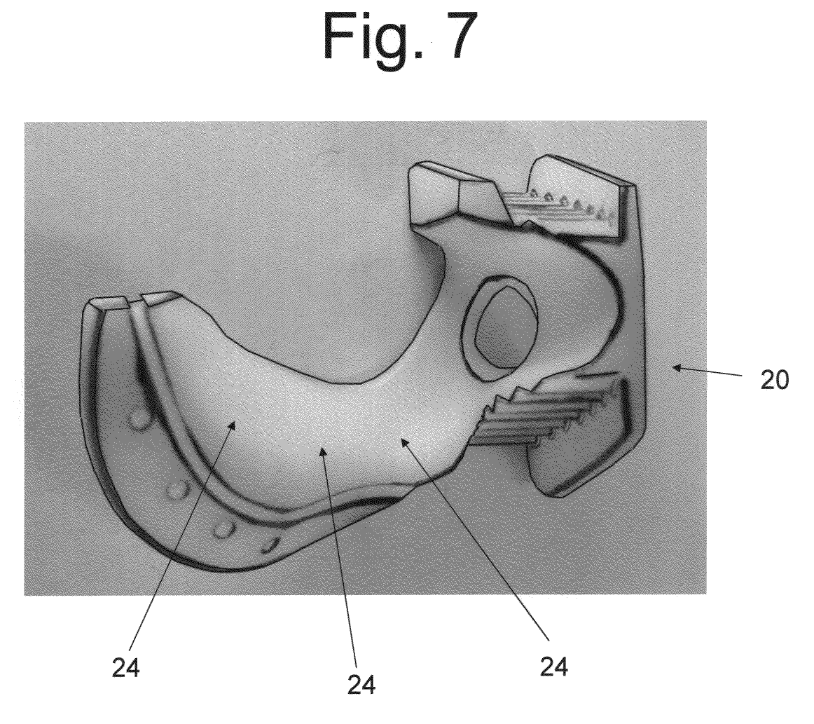

FIG. 7 depicts a kit containing therein the bite block of the invention (20), tooth brush, swabs, dental floss and a dentifrice and a disinfectant.

The suction tubing, when part of the block, may pass through the body of the bite block alone through a lateral surface or may pass through the bite block from the front. However, the tongue escape shape could also fit into a suction tube in case of extra suction ability is need. The suction tubing may be placed through the tongue guiding aperture in the mouth block, with the tubing passing between the lips and transversely through the mouth block or simply passes it through the tongue guiding hole from outside to inside.

Although the illustration here describes its shape of current invention; however, some example of variation such as different sizes and right/left version are also part of current invention.

While the bite block can be made of any material that will provide sufficient support for the jaw, preferred materials allow some deforming upon biting by the patient. Such materials include resilient plastic or rubber materials. A particularly preferred material is polystyrene which permits initial deformation to conform to the bite of the particular patient. It is also possible to use a combination of materials such as a metal core covered with resilient materials such as plastics or rubbers (natural or synthetic) of varying rigidity. When the devices are to be reused, the devices should be made of material that can be easily cleaned and sterilized. The method of sterilization may be chemical or physical (thermal, irradiation, ultrasound, etc.) and will depend on the equipment available and the material used to make the bite block. For reusable devices, a metal core covered with silicone rubber-like material or other similar material may be used.

Semi-rigid, deformable materials that deform easily upon initial contact are particularly useful for practice of the invention. Such materials deform upon initial contact because the cells are compressed in the immediate vicinity of the pressure to render the material more dense in the area compressed. Continued or additional deformation is resisted since the denser material is more rigid after deformation. Hence, once in place, the material compressed is quite resistant to further deformation. Styrenes, silicones, polyurethanes and similar materials can be made particularly for this purpose.

The prop may be constructed of a strong, long-lasting substance such as metal or rigid plastic with detachable, replaceable bite surfaces of softer, deformable materials for long-term use for patients that can not cooperate with health care workers. The device could be made with or without the groove for the suction tube. However, the device without the groove having a smooth surface would be more appropriate where suction was not needed, since a smooth surface would be easier to clean and sterilize.

The device may be provided with appropriate brushes and/or swabs, dental floss and compositions for oral care such as cleaning pastes or powders, lemon oil, and oral rinses such as Listerine or fluoride rinses in a kit form. Such a kit could be provided for bedside use.

In conclusion, according to the description disclosed above, the improved implant root provided by the present invention has the novelty and improvement that a patent should have. And the embodiments given are only for illustrating the present invention; it will be apparent to those skilled in this art that various equivalent modifications or changes without departing from the spirit of this invention, such as increasing or shape changing of the treated surfaces, shall also fall within the scope of the appended claims.

I claim:

1. A prop comprising a bite block and an elongated portion that extends from said anterior surface of said bite block wherein said elongated portion is physiologically deflected to provide a “U”-turn extension/or flange portion with a reflected concave surface to gather light illumination, seating between lateral border of the tongue and mandible dental arch.

2. The prop of claim 1 wherein the elongated flange has a concave side facing teeth and a convex side facing tongue.

3. The prop of claim 1 wherein the middle aspect of the block, a tongue guiding hole to accommodate tongue tip, dental floss or suction tubing.

4. The prop of claim 1 wherein the bite block is made of semi-rigid material.

5. The prop of claim 2 wherein the concave side is made of reflecting mirror surface.

6. The prop of claim 5 wherein said mirror surfaces are detachable and replaceable.

7. The prop of claim 1 wherein the bite block is made of a semi-rigid deformable material.

8. The prop of claim 7 wherein the bite block is made of polyurethane.

9. The prop of claim 1 wherein the bite block is made of metal covered with silicone rubber.

10. The prop of claim 1 wherein the bite surfaces are made of a material that is softer than the block.

11. The prop of claim 1 wherein said removable LED light device is build into the device for extra illumination.

12. The prop of claim 1 wherein said the tongue block to use for CAD/CAM intra-oral digital impression.

13. A kit for use in dental surgery wherein said kit contains compositions for oral care and a prop comprising a bite block having an elongated portion that extends from said anterior surface of said bite block wherein said elongated portion has concave surface to reflect the light into the oral cavity.
